(19) United States
(12) Patent Application Publication
(10) Pub. No.: US 2005/0283879 A1
(43) Pub. Date: Dec. 29, 2005

(54) System for motion sickness prevention

(76) Inventor: Tiffany Graves, Parry Sound (CA)

Correspondence Address: D.W. EGGINS, 18 DOWNSVIEW DRIVE, BARRIE, ON, L4M 4P8, CA

(21) Appl. No.: 10/875,246

(22) Filed: Jun. 25, 2004

(51) Int. Cl. A61F 9/00

(52) U.S. Cl. 2/15

(57) ABSTRACT

Vision limiting means limit the input of peripheral stimuli to the eyes of a subject, to prevent the onset of motion sickness when reading or writing during car or bus travel. The device may consist of a headband or “glasses” having two side screens or “blinkers” that extend downwardly to block off peripheral lateral visual stimuli from the user's eyes and may include corner pieces to block off the upper outer quadrants of the user's field of vision. Hook-and-loop fasteners permit ready removal and replacement. A unitary spectacle-like embodiment may be molded, or stamped from sheet plastic, with temple-pieces for looping to a user's ears.

### CROSS-REFERENCE TO RELATED APPLICATIONS

Not Applicable (N/A)

### STATEMENT REGARDING FEDERALLY SPONSORED RESEARCH OR DEVELOPMENT

N/A

### REFERENCE TO SEQUENCE LISTING/TABLE/COMPUTER PROGRAM LISTING COMPACT DISC APPENDIX

N/A

### BACKGROUND OF THE INVENTION

Many people suffer from motion sickness, when traveling in a vehicle, in which they experience feelings of nausea A particular cause for one class of sufferers of nausea is associated with their attempting to read and/or write in a vehicle while in motion. One mode of treatment consists of medication, with the taking of motion sickness pills.

In the case of motor vehicles, an alternative/addition to medication has been the use of grounding straps, to counter the build-up of static electricity in the vehicle structure.

### BRIEF SUMMARY OF THE INVENTION

In the case of motion sickness experienced primarily during car or bus travel, particularly when the person involved attempts to read and/or write, the present inventor has discovered that for many such sufferers the functioning of one's peripheral vision appears to play a significant role in the onset of that motion sickness.

To counter this effect, the present invention provides vision limiting means, to limit the input of peripheral stimuli to the eyes of a subject, when reading or writing in a moving vehicle.

The subject vision limiting means consists of light barriers, to substantially block peripheral visual inputs into the eyes of a user, and may be used in conjunction with eye-glasses.

It has been found that such peripheral blockage may be supplemented by what can be referred to as diagonal field restriction, wherein the peripheral field limitation encompasses the upper outer quadrants of the user's field of vision.

In implementing the present invention, it has been found that users who formerly were subject to motion sickness that effectively prevented them from reading while traveling in a car, due to the onset of symptoms of motion sickness, were enabled, by use of the subject vision-limiting means, to read/write without incurring the onset of such symptoms.

It has been further found, in the case of at least some users of the present system, that use of the subject vision limiting means for a brief period such as ten minutes, at the commencement of travel, then enables the use of such vision limiting means to be discontinued, without the subsequent onset of motion sickness while reading or writing in the following period of travel.

However, the converse does not appear to apply, so that if on a journey, the activities of reading or writing bring on the symptoms of motion sickness, donning of the presently disclosed vision limiting means subsequent to the onset of motion sickness will not then cure those symptoms. Thus, the subject system, and use of the presently disclosed device appears to function solely in an anticipatory, preventive mode.

### BRIEF DESCRIPTION OF THE SEVERAL VIEWS OF THE DRAWING

Certain embodiments of the invention are described by way of illustration, without limiting the scope of the invention thereto, other than as set forth in the present claims, reference being made to the accompanying drawings, wherein:

FIG. 1 is a side view of a first embodiment of the subject invention, as worn by a user;

FIG. 2 is a front-quarter perspective view of a second embodiment, upon a user;

FIG. 3 is an inverted perspective view of the FIG. 1 embodiment, with detachable “blinker” portions shown in exploded relation;

FIG. 4 is a plan view of a cut-out for a three-piece “spectacle/visor embodiment;

FIG. 5 shows the temple-pieces of the FIG. 4 embodiment; and,

FIG. 6 is a front quarter perspective view of the FIGS. 4 and 5 embodiment, as worn by a user.

### DETAILED DESCRIPTION OF THE INVENTION

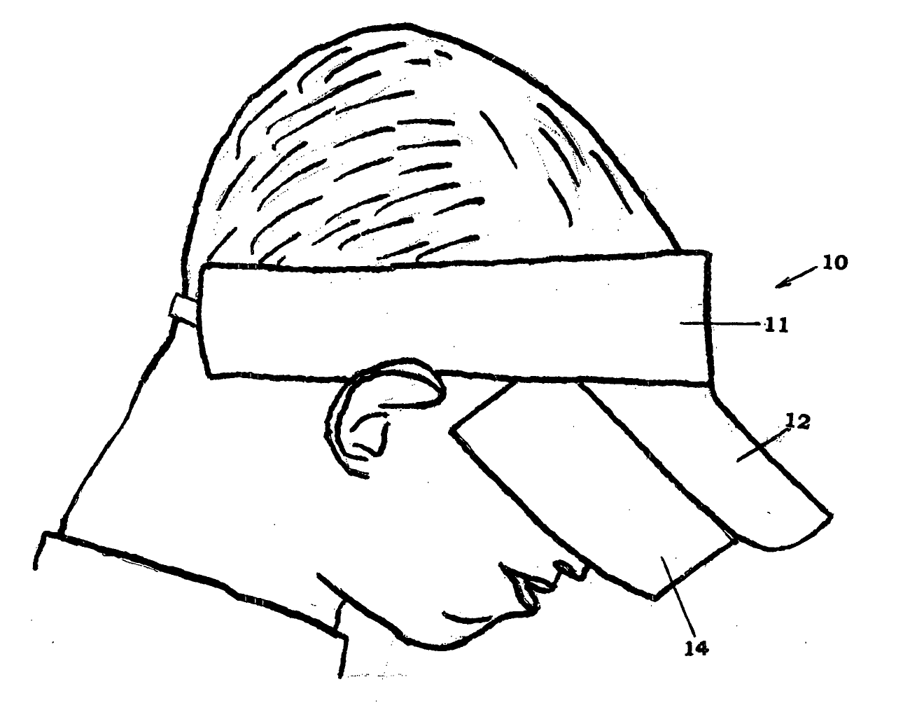

Referring to FIG. 1, a user is shown wearing a motion sickness inhibitor 10 in downwardly inclined relation as described herein, in accordance with the present invention.

The inhibitor 10 has a headband portion I 1, with a peak 12, and a pair of “blinker” sideshields 14, of which only the near-side one is seen.

The headband portion 11 may resiliently grip a wearer's head, or may include a retention band such as one adjustably secured by hook and loop fasteners like commercial Velcro (Trademark) to fully encircle the head of a wearer.

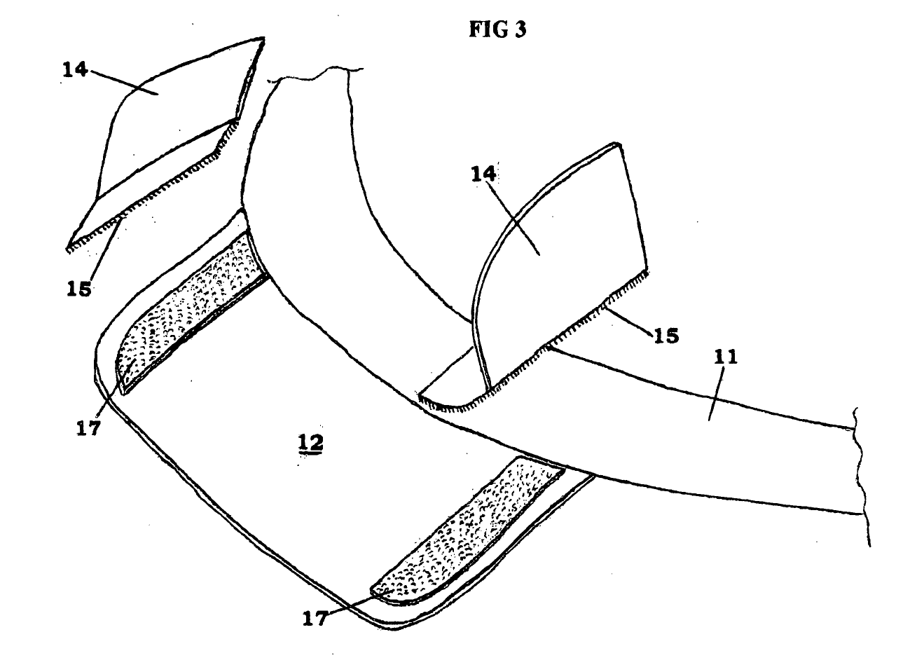

Referring to FIG. 3, the sideshields 14, 14 are detachably secured to the underside of the peak 12 by means of hook-and-loop attachments 15, 17, such as the commercially known Velcro (Trademark).

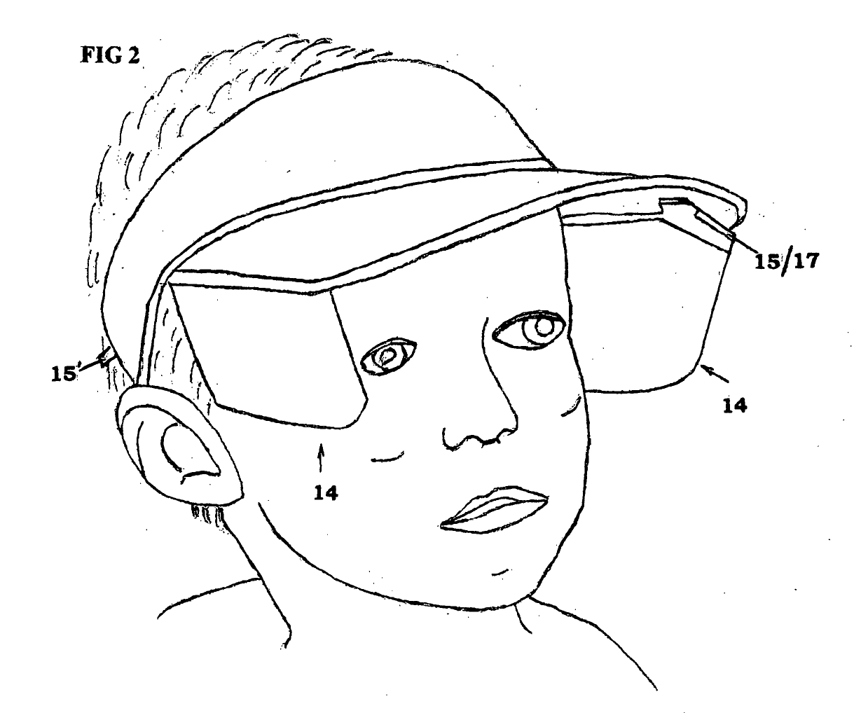

In FIG. 2, the sideshields 14 are illustrated as being detachably secured by hook and loop attachments 15. However, it will be understood that the sideshields 14 may be permanently attached to the peak 12. An adjustable hook-and-loop retention band 15′ is shown.

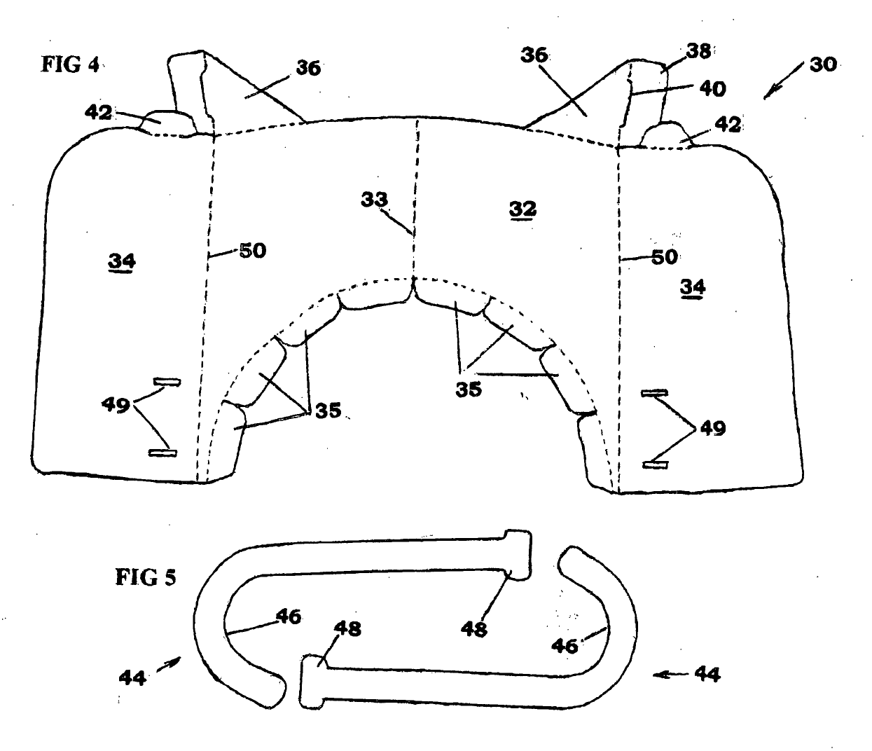

The embodiments of FIGS. 1, 2 and 3 are preferably of dull black cotton material. Referring to the FIG. 4 embodiment, the vision restrictor 30 may be a unitary molding or sheet-stamping of substantially opaque material, preferably with a matt, non-reflective surface on the ‘inside’ of the restrictor, to limit or preclude any reflective image transmission to the eyes of the user.

The restrictor 30 has a peak portion 32 with side shields 34.

The cut-lines for the stamped sheet are shown in-solid, and the resilient bend-lines, which are shown by dashed lines, may be die-formed to provide live hinges.

The peak portion 32 is illustrated as having a central bend line 33, and deformable tab portions 35, to facilitate conforming of the peak portion 32 to the brow of a wearer. Triangular corner pieces 36 each have a hinged tab portion 38, with a perforated slit 40. The sideshields 34 each has a hinged tab portion 42.

Shown in FIG. 5, temple pieces 44 each include an earpiece 46 and anchor protrusions 48. The temple pieces 44 are secured by way of slots 49 to the sideshields 34.

To assemble the sideshields 34 with the corner pieces 36, the sideshields 34 are bent about their hingelines 50, to be at rightangles to the peak portion 32. Then the corner pieces 36 are bent at rightangles to the peak portion 32, with the tab portions 42 being entered into the respective slit 40. The adjacent tab portions 38 are then bent into locking engagement with respective sideshield 34; and the tab portions 42 bent into flush relation with the corner pieces 36.

The thus-assembled vision restrictor 30 is then adjusted to the forehead of the user, being secured to the ears of the user by way of the temple pieces 44.

The substantially opaque vision restrictor 30, in that no visual image is transmitted through its elements, may comprise a thin, semi-translucent plastic, which permits the transmission of light, but blocks image transmission, and the inner surface of which is substantially non-reflective.

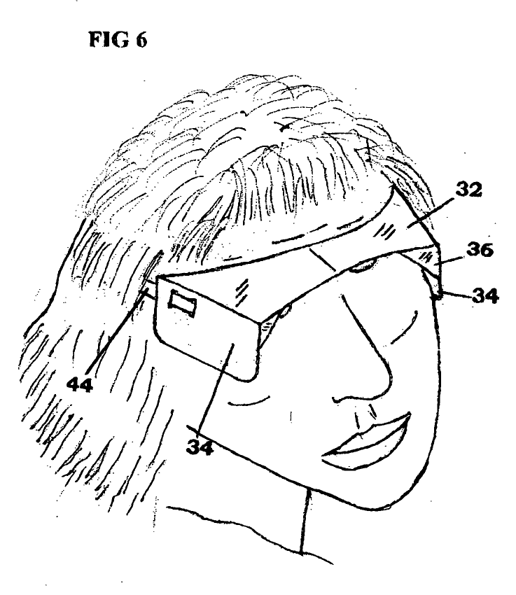

FIG. 6 illustrates the FIGS. 4 and 5 embodiment assembled, as it might appear in use.

In a preferred method of use, prior to commencing reading or writing, and wearing the vision restrictor with the side panels oriented straight up and down, the user should be looking downwardly, so as to inhibit looking around or outside the vehicle. The optimum downward inclination of the head and eyes is advised, for best results. After commencement of use, the inhibition against looking around or outside the vehicle should be maintained for about ten minutes.

Test subjects of large girth, whose physiognomy prevents them from positioning their reading material in a favourable downward position may require the provision of field-of-vision inhibitors that are specifically tailored to extend the inhibited field to meet their particular needs. This may include the adoption of modified peaks for the headband

The subject field-of-vision restrictor is not intended to deal with motion sickness other than nausea caused by attempting to read and/or write in a vehicle while in motion.

1. Vision limiting means to limit the visual input of peripheral stimuli to the eyes of a user, consisting of a browpiece, sideshield portions connected with the browpiece, and support means for locating the vision limiting means in predetermined positioned relation on the head of a user, in use to limit the potential field of vision of the user, to substantially preclude peripheral visual inputs.

2. The vision limiting means as set forth in claim 1, including diagonal field restriction means, wherein the peripheral field limitation encompasses the upper outer quadrants of the user's field of vision.

3. The vision limiting means as set forth in claim 1, having a headband with a visor portion; said sideshield portions extending downwardly from the visor portion, to substantially block the input of lateral peripheral images to the eye of a user.

4. The vision limiting means as set forth in claim 3, wherein said sideshield portions are removably secured to said visor portion.

5. The vision limiting means as set forth in claim 4, wherein said sideshield portions are secured to said visor portion by means of hook and eye attachment means.

6. The vision limiting means as set forth in claim 3, including diagonal field restriction means, to substantially exclude peripheral visual inputs to the upper outer quadrants of the user's field of vision.

7. The vision limiting means as set forth in claim 1, wherein said browpiece, and said sideshield portions are unitary; and wherein said support means for locating the vision limiting means in predetermined positioned relation on the head of a user, consist of a pair of temple pieces to engage the ears of said user.

8. The vision limiting means as set forth in claim 7, being of sheet material, and having live hinge portions connecting adjoining portions, to permit relative folding of said adjoining portions, and tab means interconnecting said adjoining portions in mutually engaging relation.

9. The vision limiting means as set forth in claim 1, being of dark fabric material.

10. The vision limiting means as set forth in claim 9, said material having at least one dull, substantially non-reflective surface.

11. Vision limiting means in the form of a visor, for limiting the peripheral field of vision of a user, consisting of a planar sheet of substantially opaque material having a peak portion with a forward edge and a rear edge; side shield portions on each side of said peak portion, in adjoining relation therewith; said peak portion rear edge being curved, with adjoining flap portions; two triangular corner pieces projecting forwardly of said forward edge, each corner piece having a first tab portion extending along a side edge thereof, with a perforating slit extending between each said corner piece and said first tab portion; each said flap portion having a forward projecting second tab portion hinged thereto; a pair of temple pieces removably attached to said side shield portions; and hinge means interconnecting selected ones of said visor portions, whereby in use, said side flaps are bent downwardly in mutual parallel relation; said corner pieces are bent downwardly, whereby said second tab portions each enter a said perforating slit, to secure said side flaps in secured relation with said corner pieces, to enable the wearing of said visor by a user, in blocking relation with the peripheral field of vision of said user.

12. The vision limiting means as set forth in claim 11, said material having at least one dull, substantially non-reflective surface.
